(19) United States
(12) Patent Application Publication
(10) Pub. No.: US 2007/0025967 A1
(43) Pub. Date: Feb. 1, 2007

(54) Zap protein and related compositions and methods

(76) Inventors: Stephen P. Goff, Tenafly, NJ (US); Guanxia Gao, Beijing (CN)

Correspondence Address: COOPER & DUNHAM, LLP, 1185 AVENUE OF THE AMERICAS, NEW YORK, NY, 10036, US

(21) Appl. No.: 10/568,396

(22) PCT Filed: Aug. 12, 2004
(86) PCT No.: PCT/US04/26162

§ 371 Date: Aug. 31, 2006

Related U.S. Application Data
(60) Provisional application No. 60/494,764, filed on Aug. 13, 2003.

(51) Int. Cl. A61K 48/00; A61K 38/48; C12Q 1/70; C12Q 1/68; C07H 21/04; C12P 21/06; C12N 9/12

(52) U.S. Cl. 424/93.2; 435/6; 435/69.1; 435/194; 435/320.1; 435/325; 424/94.5; 536/23.2; 435/5

(57) ABSTRACT

This invention provides an isolated ZAP protein and methods and articles of manufacture for increasing resistance to a virus in a subject or mammalian cell. The instant methods and articles are based on the use the ZAP protein to increase such resistance to a virus.

This invention was made with support under United States Government Public Health Service Grant CA 30488. Accordingly, the United States government has certain rights in this invention.

Throughout this application, various publications are, referenced. Full citations for these publications may be found immediately preceding the claims. The disclosures of these publications are hereby incorporated by reference into this application in order to more fully describe the state of the art as of the date of the invention described and claimed herein.

### BACKGROUND OF THE INVENTION

Vertebrate cells have evolved a number of defense mechanisms to prevent or inhibit viral replication after an infection. A remarkable array of such antiviral proteins are induced by interferon (2), including: PKR, a double-stranded RNA-dependent kinase that phosphorylates eIF-2α and shuts down translation (3); the Mx proteins, GTPasses that block viral gene expression by unknown mechanismsm (4); and oligoA synthetases (5), producing 2′,5′-oligodenylates (6) that activate Rnase L to degrade both mRNAs. In some cases the antiviral state involves a drastic shutoff of host functions In other cases, there is a more specific block to viral replication or gene expression. While many parallel pathways have been uncovered, it is likely that still more antiviral proteins remain to be found.

### SUMMARY OF THE INVENTION

This invention provides an isolated ZAP protein.

This invention further provides an isolated nucleic acid which encodes a ZAP protein.

This invention further provides an expression vector comprising a nucleic acid sequence encoding a ZAP protein.

This invention further provides a method for increasing the amount of ZAP protein in a mammalian cell which comprises contacting the cell with a ZAP protein under conditions permitting entry of the ZAP protein into the cell, so as to thereby increase the amount of ZAP protein in the mammalian cell.

This invention further provides a method for increasing the expression of ZAP protein in a mammalian cell which comprises introducing into the cell an expression vector comprising a nucleic acid sequence encoding a ZAP protein, so as to thereby increase ZAP protein expression in the mammalian cell.

This invention further provides a method for increasing resistance to a virus in a mammalian cell which comprises contacting the cell with a ZAP protein specific for that virus under conditions permitting entry of the ZAP protein into the cell, so as to thereby increase resistance to the virus in the cell.

This invention further provides a method for increasing resistance to a virus in a mammalian cell which comprises introducing into the cell an expression vector comprising a nucleic acid sequence encoding a ZAP protein specific for that virus, so as to thereby increase resistance to the virus in the mammalian cell.

This invention further provides a method for increasing the amount of ZAP protein in a subject's cells which comprises administering to the subject an amount of ZAP protein effective to increase the amount of ZAP protein in the subject's cells.

This invention further provides a method for increasing resistance to a virus in a subject which comprises administering to the subject an amount of ZAP protein specific for that virus effective to increase the amount of ZAP protein in the subject's cells, so as to thereby increase resistance to the virus in the subject.

This invention further provides a method for determining whether an agent increases ZAP protein expression in a mammalian cell which comprises: (a) contacting the cell with the agent under conditions permitting ZAP protein expression; (b) determining the resulting amount of ZAP protein expression in the cell; and (c) comparing the amount of expression determined in step (b) with the amount of ZAP protein expression determined in the absence of the agent, whereby a greater amount of ZAP protein expression in the presence of the agent relative to that in the absence of the agent indicates that the agent increases ZAP protein expression in a mammalian cell.

This invention further provides a method for determining whether an agent increases resistance to a virus in a mammalian cell, which comprises: (a) contacting the agent with a mammalian cell having introduced thereto an expression vector comprising a nucleic acid sequence corresponding to the virus operatively linked to a reporter sequence whose expression in a mammalian cell gives rise to a detectable signal, wherein RNA corresponding to the virus is known to be degraded by a ZAP protein present in the cell; (b) determining the amount of signal produced in the cell by the reporter sequence after contact with the agent; and (c) comparing the amount of signal determined in step (b) to that produced in the absence of the agent, whereby the amount of signal produced in the presence of the agent being less than that produced in the absence of the agent indicates that the agent increases resistance to the virus in the cell.

This invention further provides a composition comprising a ZAP protein and a pharmaceutically acceptable carrier.

This invention further provides a composition comprising an expression vector comprising a nucleic acid sequence encoding a ZAP protein, and a pharmaceutically acceptable carrier.

This invention further provides an article of manufacture comprising a packaging material having therein a ZAP protein and a label indicating a use for the ZAP protein for increasing resistance to a virus in a subject.

Finally, this invention provides an article of manufacture comprising a packaging material having therein an expression vector comprising a nucleic acid sequence encoding a ZAP protein, and a label indicating a use for the expression vector for increasing resistance to a virus in a subject.

### BRIEF DESCRIPTION OF THE FIGURES

FIG. 1 Schematic representation of pBabe-HAZ construct, MLV viral RNA packaging signal; HA, hemagglutinin epitope tag; EcoRI-NotI, linker sequence containing EcoRI and NotI sites; LoxP, LoxP as sequence for recognition by Cre recombinase.

FIG. 2A Resistance of NZAP-zeo expressing cells to virus infection. Cre recombinase was stably introduced into 11D3 cells by cotransformation with pGk-puro. Five puromycin-resistant clones were expanded and tested for resistance to Eco-Luc virus (lower panel). Deletion of NZAP-zeo DNA in each cell line was monitored by PCR (upper panel).

FIG. 2B The NZAP-zeo fragment was recovered and reintroduced into naïve Rat2 cells. These cells were compared with the original L1D3 cells for the resistance to Eco-Luc virus. Rat2, wild type Rat2 cells; L1D3 cells isolated from the screening; Rat2-HAZ, Rat2 cells expressing pBabe-HAZ empty vector; Rat2-NZAP-zeo, Rat 2 cells expressing recovered pBabe-Nzap-zeo.

FIG. 2C Cells were infected with wild-type MuLv at low multiplicity. The culture supernatants were harvested 2, 4, 6, 8 and 10 days after infection and were analyzed for reverse transcriptase (RT) activity to measure the spread of the virus. RT signals were quantified by Phospoimager and plotted.

FIG. 3A Schematic representation of rZAP and related sequences. The cDNA fragment recovered from L1D3 cells. ATG, start codon; TAG stop codon in 5′UTR; NZAP-zeo, open reading frame of rZAP and Zeo fusion protein.

FIG. 3B Blast search of Genbank database with NZAP fragment identified two mouse EST clones (mEST995 and mEST896) that have high sequence similarity to rZAP. These two clones were obtained from ATTC and sequences. The 3′ end sequence of mEST995 was used to design a primer to PCR amplify full-length rZAP from Rat2 cDNA library. Deduced amino acid sequences are compared schematically. The numbers of amino acids of each coding sequence are indicated. The positions of four CCCH finger motifs are indicated by black boxes and the sequences of the motifs in rZAP are shown.

FIG. 3C Northern blot of tissue mRNAs of rat probed with rZAP cDNA.

FIG. 4A Analysis of the position of the block to virus infection. Cells were infected with either Eco-Luc virus (upper panel) or Eco-GFP virus (lower panel) at various dilutions. Viral DNA was extracted 24 h post-infection and detected by using PCR using primers that specifically amplify minus strand strong stop DNA or LTR-LTR circular junction sequence, respectively. The positions of the PCR products are indicated.

FIG. 4B Cells were transiently transfected with DNA of the MLV viral vector expressing luciferase (MLV-Luc), or control vector expressing luciferase under CMV promoter (CMV-Luc). Forty-eight hours after transfection, cells were lysed and luciferase activity was measured.

FIG. 4C Analysis of viral RNA levels in infected cells. Cells infected with undiluted Eco-Luc virus, and forty-eight hours post infection total RNA, nuclear RNA and cytoplasmic RNA fractions were isolated. 20 micrograms of RNA from each fraction was resolved by electropheresis in agarose gel and then transferred to a Nylon membrane. The membrane was probed with P32-labeled luciferase DNA and exposed to X-ray film (top panel). The same membrane was stipped and re-probed with 32P-labeled GAPDH DNA aand exposed to X-ray film (middle panel). To monitor the integrity of the RNA samples, the gel was stained with ethidium bromide (bottom panel) before transfer to the membrane. The relative RNA levels were quantitated by Phosphoimager and plotted. The positions of each RNA are indicated. E, Rat2-empty vector control cells; Z, Rat2-NZAP-zeo cells.

FIG. 4D Biological activity of various forms of rZAP. Rat2 cells stably expressing the empty vector (black bar) or NZAP-zeo (grey bar) were transiently transfected with an empty vector DNA, a plasmid expressing the full-length ZAP (pZAP-myc), or a plasmid expressing a fragment (NZAP-myc). The cells were then challeneged with MLV-Luc virus and following forty-eight hour lysates, were assayed for luciferase. The full-length ZAP inhibited infection, whicle the ZAP fragment relieved the inhibition caused by NZAP-zeo.

FIG. 5A Amino acid sequence of the ZAP protein.

FIG. 5B Nucleic acid sequence of ZAP cDNA.

### DETAILED DESCRIPTION OF THE INVENTION

Definitions

As used in this application, except as otherwise expressly provided herein, each of the following terms shall have the meaning set forth below.

As used herein, “administering” shall mean delivering in a manner which is effected or performed using any of the various methods and delivery systems known to those skilled in the art. Administering can be performed, for example, intravenously, orally, via implant, transmucosally, transdermally, intramuscularly, or subcutaneously. “Administering” can also be performed, for example, once, a plurality of times, and/or over one or more extended periods.

As used herein, “agent” shall include, without limitation, an organic compound, a nucleic acid, a polypeptide, a lipid, and a carbohydrate. Agents include, for example, agents which are known with respect to structure and/or function, and those which are not known with respect to structure or function.

As used herein, “conditions permitting entry of the ZAP protein into the cell” include, for example, physiological conditions.

As used herein, “host cells” include, but are not limited to, bacterial cells, yeast cells, fungal cells, insect cells, and mammalian cells. Mammalian cells can be transfected by methods well-known in the art such as calcium phosphate precipitation, electroporation and microinjection.

As used herein, “increasing resistance” to a virus shall mean inhibiting the replication of the virus in a cell infected therewith. In one embodiment, this inhibition is characterized by a reduction in mRNA encoding viral proteins.

As used herein, “mammalian cell” shall mean any mammalian cell. Mammalian cells include, without limitation, cells which are normal, abnormal and transformed, and are exemplified by neurons, epithelial cells, muscle cells, blood cells, immune cells, stem cells, osteocytes, endothelial cells and blast cells.

As used herein, “nucleic acid” shall mean any nucleic acid molecule, including, without limitation, DNA, RNA and hybrids thereof. The nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA).

As used herein, “pharmaceutically acceptable carriers” are well known to those skilled in the art and include, but are not limited to, 0.01-0.1 M and preferably 0.05 M phosphate buffer or 0.8% saline. Additionally, such pharmaceutically acceptable carriers can be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions and suspensions, including saline and buffered media. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like.

As used herein, “protein” and “polypeptide” are used equivalently, and each shall mean a polymer of amino acid residues. The amino acid residues can be naturally occurring or chemical analogues thereof. Polypeptides and proteins can also include modifications such as glycosylation, lipid attachment, sulfation, hydroxylation, and ADP-ribosylation.

As used herein, “protein instability” shall mean the propensity, with respect to a protein, to undergo degradation or other modification adversely affecting the function of the protein.

As used herein, “reporter sequence” shall mean a nucleotide sequence whose expression in a mammalian cell gives rise to a detectable signal.

As used herein, RNA “corresponding” to a virus includes, without limitation, RNA normally found within the virus (as in the case of a retrovirus), and mRNA produced by a cell infected with the virus using DNA from the virus as a template.

As used herein, “subject” shall mean any animal, such as a non-human primate, mouse, rat, guinea pig, dog, cat, or rabbit.

As used herein, “vector” shall mean any nucleic acid vector known in the art. Such vectors include, but are not limited to, plasmid vectors, cosmid vectors, and bacteriophage vectors.

As used herein, “virus” shall mean any of a large group of microscopic infective agents that are regarded either as the smallest microorganisms or extremely complex molecules and are composed of a protein coat surrounding an RNA or DNA core of genetic material and are capable of growth and multiplication in living cells.

As used herein, “WWE” shall mean a globular protein domain in proteins involved in protein-protein interactions in ubiquitin and ADP-ribose conjugation systems.

As used herein, “ZAP” and “ZAP protein” are used synonymously, and each shall mean a mammalian protein which (a) comprises Four CCCH-type zinc finger motifs, (b) binds to RNA corresponding to at least one type of virus (“target virus”), and (c) when present in a mammalian cell infected with a target virus, binds to RNA corresponding to the target virus, so as to inhibit replication of the target virus in the cell.

As used herein, a ZAP protein “specific” for a virus shall mean a ZAP protein known to cause the degradation of RNA corresponding to that virus.

### EMBODIMENTS OF THE INVENTION

This invention provides an isolated ZAP protein. In one embodiment, the protein is a human protein. In a further embodiment, the protein is a rat protein. In another embodiment, the protein is a mouse protein. In yet another embodiment, the protein comprises the amino acid sequence set forth in SEQ ID NO:1

The instant protein can have deleted from it a region which causes protein instability. In one embodiment, the deleted region is the WWE region.

This invention further provides an isolated nucleic acid which encodes a ZAP protein. In one embodiment, the nucleic acid is DNA. In a further embodiment, the DNA is cDNA. In another embodiment, the cDNA comprises the nucleic acid sequence set forth in SEQ ID NO: 2. In yet another embodiment, the DNA is genomic DNA. In yet another embodiment, the nucleic acid is RNA.

In one embodiment, the nucleic acid encodes a human ZAP protein. In yet another embodiment, the nucleic acid encodes a rat ZAP protein. In yet another embodiment, the nucleic acid encodes a mouse ZAP protein.

The instant nucleic acid can encode a ZAP protein which has deleted from it a region which causes protein instability. In one embodiment, the deleted region is the WWE region.

The instant nucleic acid can be labeled with a detectable marker. In one embodiment, the detectable marker is a radioactive label, a calorimetric marker, a luminescent marker or a fluorescent marker.

This invention further provides an expression vector comprising a nucleic acid encoding a ZAP protein. In one embodiment, a host vector system comprises the expression vector and a suitable host cell. In a further embodiment, the host cell is a eukaryotic, bacterial, insect or yeast cell. In another embodiment, the host cell is a mammalian cell.

This invention further provides a method for increasing the amount of ZAP protein in a mammalian cell which comprises contacting the cell with a ZAP protein under conditions permitting entry of the ZAP protein into the cell, so as to thereby increase the amount of ZAP protein in the mammalian cell.

This invention further provides a method for increasing the expression of ZAP protein in a mammalian cell which comprises introducing into the cell an expression vector comprising a nucleic acid sequence encoding a ZAP protein, so as to thereby increase ZAP protein expression in the mammalian cell.

In one embodiment of the instant method, the method comprises the step of detecting the increase in ZAP protein expression by detecting a difference in the amount of ZAP-protein encoding mRNA in the mammalian cell before and after introduction of the expression vector into the cell.

This invention further provides a method for increasing resistance to a virus in a mammalian cell which comprises contacting the cell with a ZAP protein specific for that virus under conditions permitting entry of the ZAP protein into the cell, so as to thereby increase resistance to the virus in the subject.

In one embodiment of the instant method, the mammalian cell is a human cell. In another embodiment, the virus is an alpha virus. In yet another embodiment, the virus is West Nile virus.

This invention further provides a method for increasing resistance to a virus in a mammalian cell which comprises introducing into the cell an expression vector comprising a nucleic acid sequence encoding a ZAP protein specific for that virus, so as to thereby increase resistance to the virus in the mammalian cell.

In one embodiment of the instant method, the mammalian cell is a human cell. In another embodiment method, the virus is an alpha virus. In yet another embodiment, the virus is West Nile virus.

This invention further provides a method for increasing the amount of ZAP protein in a subject's cells which comprises administering to the subject an amount of ZAP protein effective to increase the amount of ZAP protein in the subject's cells. In one embodiment of the instant method, the subject is human.

This invention further provides a method for increasing resistance to a virus in a subject which comprises administering to the subject an amount of ZAP protein specific for that virus effective to increase the amount of ZAP protein in the subject's cells, so as to thereby increase resistance to the virus in the subject.

In one embodiment of the instant method, the subject is human. In another embodiment, the virus is an alpha virus. In yet another embodiment, the virus is West Nile virus.

This invention further provides a method for determining whether an agent increases ZAP protein expression in a mammalian cell which comprises: (a) contacting the cell with the agent under conditions permitting ZAP protein expression; (b) determining the resulting amount of ZAP protein expression in the cell; and (c) comparing the amount of expression determined in step (b) with the amount of ZAP protein expression determined in the absence of the agent, whereby a greater amount of ZAP protein expression in the presence of the agent relative to that in the absence of the agent indicates that the agent increases ZAP protein expression in a mammalian cell.

In one embodiment of the instant method, the method comprises determining the resulting amount of ZAP protein expression by determining the amount of ZAP protein-encoding mRNA in the mammalian cell. In another embodiment of the instant method, the agent is a ZAP protein having deleted from it a region which causes protein instability. In yet another embodiment of the instant method, the deleted region is the WWE region.

This invention further provides a method for determining whether an agent increases resistance to a virus in a mammalian cell, which comprises: (a) contacting the agent with a mammalian cell having introduced thereto an expression vector comprising a nucleic acid sequence corresponding to the virus operatively linked to a reporter sequence whose expression in a mammalian cell gives rise to a detectable signal, wherein RNA corresponding to the virus is known to be degraded by a ZAP protein present in the cell; (b) determining the amount of signal produced in the cell by the reporter sequence after contact with the agent; and (c) comparing the amount of signal determined in step (b) to that produced in the absence of the agent, whereby the amount of signal produced in the presence of the agent being less than that produced in the absence of the agent indicates that the agent increases resistance to the virus in the cell.

In one embodiment of the instant method, the agent is a ZAP protein having deleted from it a region which causes protein instability. The deleted region can be, for example, the WWE region. In a further embodiment of the instant method, the reporter sequence is lacZ. In another embodiment, the virus is an alpha virus. In yet another embodiment, the virus is West Nile Virus.

This invention further provides a composition comprising a ZAP protein and a pharmaceutically acceptable carrier.

This invention further provides a composition comprising an expression vector comprising a nucleic acid sequence encoding a ZAP protein, and a pharmaceutically acceptable carrier.

This invention further provides an article of manufacture comprising a packaging material having therein a ZAP protein, and a label indicating a use for the ZAP protein for increasing resistance to a virus in a subject.

Finally, this invention provides an article of manufacture comprising a packaging material having therein an expression vector comprising a nucleic acid sequence encoding a ZAP protein, and a label indicating a use for the expression vector for increasing resistance to a virus in a subject.

This invention is illustrated in the Experimental Details section which follows. This section is set forth to aid in an understanding of the invention but is not intended to, and should not be construed to, limit in any way the invention as set forth in the claims which follow thereafter.

Experimental Details

Synopsis

Viral replication requires that the incoming virus successfully target a site of replication, express viral mRNAs and proteins, and assemble progeny virions. Cells have evolved several mechanisms by which they can inhibit viral replication. To identify novel viral inhibitors, large mammalian cDNA libraries for any genes which could protect cells from infection by a genetically marked retrovirus were generated and screened. Virus resistant cells were selected from pools of transduced clones, and an active antiviral cDNA was recovered from one such line. The gene encoded a CCCH-type zinc finger protein dubbed “ZAP”. Expression of the gene in rat fibroblasts caused a profound and specific loss of the viral mRNAs from the cytoplasm without affecting the levels of the nuclear mRNAs.

Methods and Discussion

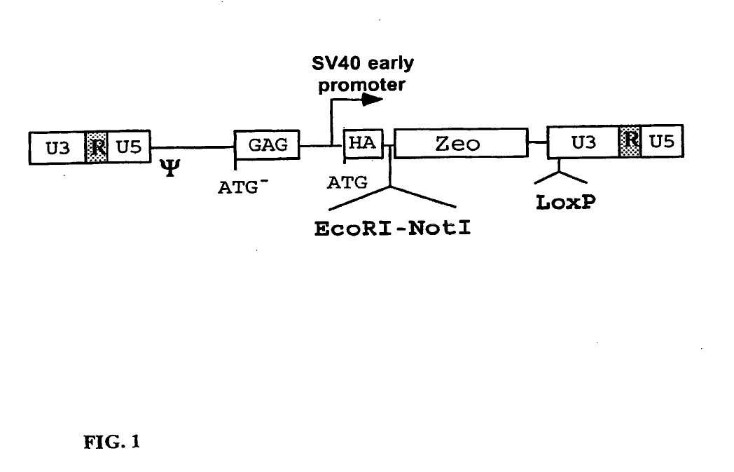

A library of expressed cDNAs was constructed in a retroviral vector, termed pBabe-HAZ. This vector was constructed by making modifications to pBabe-puro. The EcoRI and NotI sites in pBabe-puro were sequentially removed by digestion, polishing of the ends by Klenow polymerase and ligation. The puromycin resistance gene was replaced by a zeocin resistance gene prepared by PCR with various components built in the primers. The up stream primer (5′ATAAGCTTGCCACCATGGCTTSTCCSTSTGSTGTTC CAGATATGCTGAATTCGGCGGCCGCGCCAAGTTGACCAGTGC-3′) contained the HindIII cloning site, kozak consensus sequence, ATG start codon, HA tag and ECORI/NotI linker sequences, with HA tag fused to the zero gene. The downstream primer 5′ATATCGATTCAGTCCTGCTCCTCGGC-3′) contained the ClaI cloning site. The Lox P sequence was inserted by annealing two oligonucleotides (5′CTAGATAACTTCGTATAATG TATGCTATACGAAGTTAT-3′) and (5′CTAGATAACTTCGTATAGCATACAT TATACGAAGTTAT-3′) and ligating the product into the unique Nhel site in the U3 region of the 3′LTR. To minimize the background of parental vector in the cDNA library, a 1-kb stuffer sequence was inserted between the EcoRI and NotI sites to disrupt the HA-Zeo open reading frame. cDNAs were then used to replace the stuffer. (FIG. 1A). Randomly primed cDNAs from wild-type Rat2 fibroblasts were inserted into the vector under the control of a constitutive promoter, such that a hemagglutinin (HA) epitope tag was fused at the 5′ end, and a Zeocin resistance gene at the 3′ end, encoding HA-orf-Zeo fusion proteins. RNA extracted from RAT2 cells with RNA extraction kit (Amersham-Pharmacia) following the manufacturer's instructions. cDNA was synthesized from the mRNA and cDNA synthesis kits (Stratagene) following the manufacturer's instructions with the following modifications: a) NotI-oligo(dT) primer (Amersham-Pharmacia) was used to replace XhoI-oligo(dt); b) for each reaction, 15 ug, instead of 5 mg of mRNA was used as template to favor the synthesis of short cDNA fragments. The cDNA was cloned into pBabe-Haz digested with EcoRI and NotI and the reaction products were used to transform Electromax bacteria by electroporation. A LoxP sequence, the site recognized by the Cre recombinase, was inserted into the U3 region of the 3′ Long Terminal Repeat (LTR), which is duplicated during reverse transcription of the vector so that LoxP sited are present in both LTRs of the provirus after integration. These sites are positioned such that the provirus can be excised from the genome by the Cre recombinase. The complexity of the library was 2×10⁷, with inserts ranging in size from 0.2 kb to 3 kb.

A scheme was developed to identify antiviral genes in the library (outlined in FIG. 1B). Aliquots of the library DNAs were used to transform 293T cells along with DNAs encoding the Moloney MuLV Gag-Pol proteins and the VSV G envelope protein, producing 20 pools of pseudotyped transducing viruses. To generate the library of transducing viruses, a mixture of three DNAs, the pBabe-HAZ-Rat2 cDNA library, the Gag-Pol gpt plasmid, and the pMDG plasmid for the production of VSV-G, was transiently introduced into 293T cells by calcium phosphate-mediated transformation. The culture supernatant was collected 60 hours after transfection and used to infect Rat2 cells. While an antiviral gene in the library might cause loss of the corresponding transducing virus, it was hypothesized that the high and transient expression in 293T cells would override its activity. The transducing viruses were used to infect thymidine kinase-negative (TK−) Rat2 cells, and recipient clones were selected by culture in zeocin. Each clone in the pooled cells overexpressed a single member of the cDNA library.

To identify any genes in the pooled cells that conferred retrovirus resistance, we made use of a powerful selection for virus-resistant cells (14). The pools of transduced TK− Rat2 cells were challenged by repeated infection with both ecotropic and amphotropic retroviruses expressing the TK gene from the viral promoter. The bulk of the virus-sensitive cells, now having become TK positive, were then killed by growth in the toxic thymidine analogue trifluorothymidine (TFT), and rare TK-negative clones were recovered as candidate virus-resistant cells. Out of a total of 5×10⁵ transduced lines put through the selection, approximately 200 TFT-resistant clones were isolated. Retests of each of the clones showed that one, line L1D3, was dramatically resistant to virus infection. L1D3 cells were approximately 30-fold less sensitive than the parent cells to viruses carrying a luciferase reporter (Eco-Luc virus).

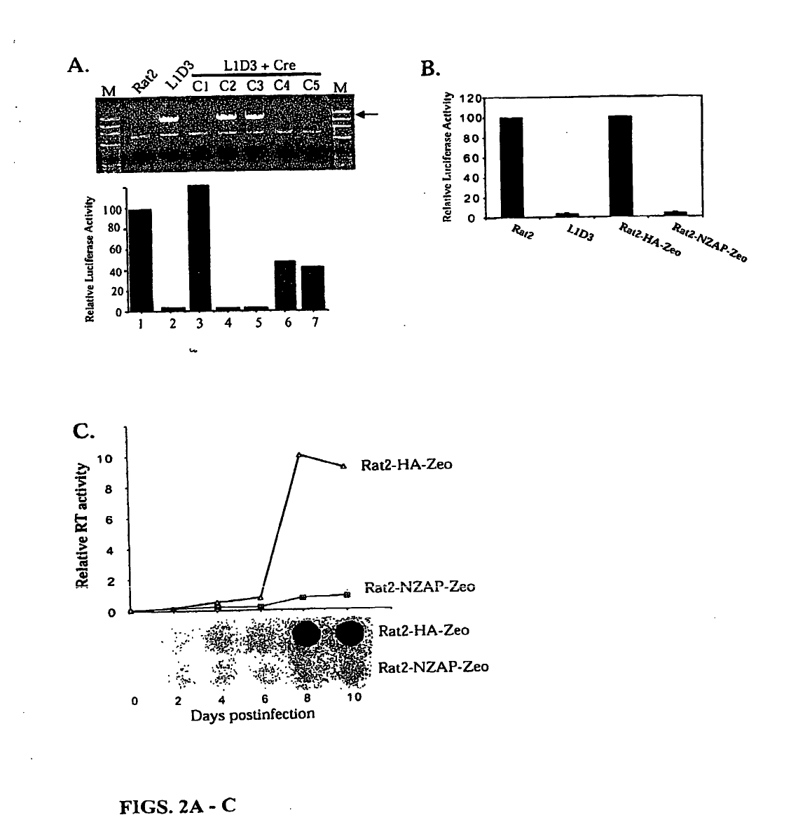

To confirm that the cDNA insert in the L1D3 line was responsible for the resistance, the cells were transfected with a construct expressing the Cre recombinase to induce the excision of the provirus at the LoxP sites and the loss of the cDNA. Five stable transfectants were analyzed for their resistance to infection by Eco-Luc virus and for the presence or loss of the cDNA by PCR amplification of the genomic DNA (FIG. 2A). To generate Ecotropic MLV-luciferase virus, retroviral vector pSRαL-Luc was stably introduced into GP+E86 producer cells by cotransformation with pSV2-Neo. Approximately 100 G418-resistant clones were individually tested for release of Eco-Luc virus. The clone that produced the highest titer of virus was explained and used for subsequent experiments. Those clones from which the cDNA was excised were all susceptible to infection, while the clones that retained the cDNA were all resistant. These experiments confirmed that the cDNA was indeed necessary for the virus resistance of the L1D3 line.

The cDNA insert in L1D3 was recovered from the genomic DNA by PCR amplification and cloned. To recover the cDNA insert from the L1D3 cell line, 1 mg of genomic DNA was used as a template in a 50 ml PCR reaction with the Expand High Fidelity PCR kit under the following conditions: 10 cycles of 94° C. for 15 seconds, 50° C. for 30 seconds, 72° C. for 60 seconds each cycle, followed by 20 cycles of 94° C. for 15 seconds, 55° C. for 30 seconds, 72° C. for 60+5 seconds each cycle. The sense primer was 5′GCTTATCCATATGATGTTCCAGATT-3′, and the antisense primer was CZAP-ap-AP (5′ATATAGGCGGCCGCCCTCTGGACCTCTTCTCTTC-3′). To confirm that the cDNA was sufficient to induce virus resistance, the cDNA was recloned into the pBabe-HAZ vector and then reintroduced into naive Rat2 cells. Cells expressing the cDNA were again 30-fold resistant to the Eco-Luc virus as compared to the parental cells or cells carrying the empty vector (FIG. 2B). Thus, the expression of the cDNA was sufficient to establish viral resistance.

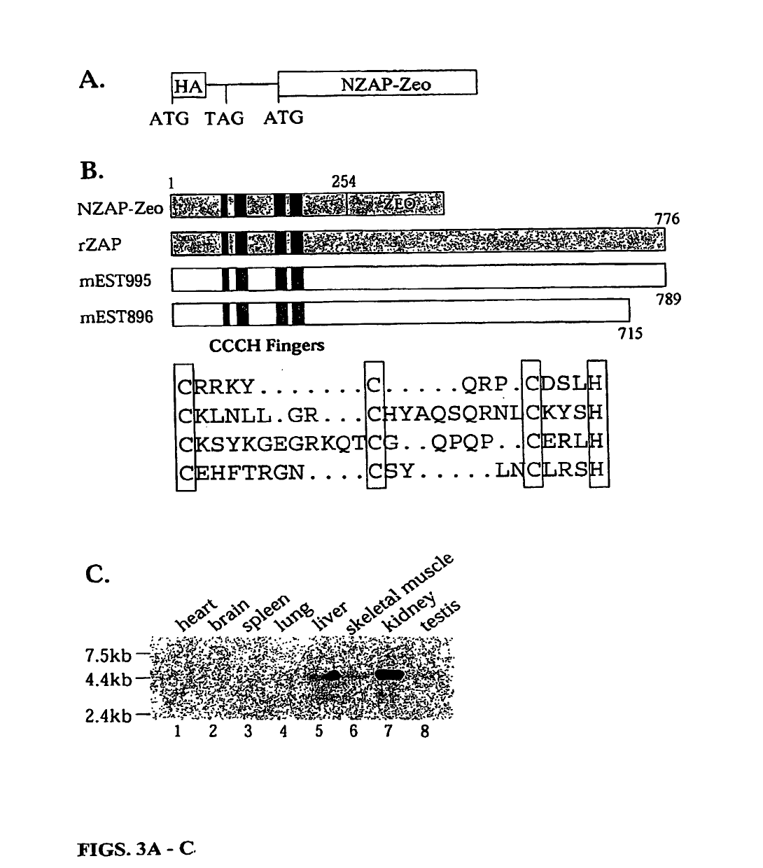

The DNA sequence of the insert revealed a single long open reading frame of 254 codons fused to the zeocin resistance gene at its 3′ end (FIG. 3A). The insert contained a long 5′ untranslated region (UTR) and the ORF was not fused to HA at the 5′ end; translation of the HA sequence terminated in the UTR and expression of the protein required translation initiation at an AUG codon at the start of the ORF, in the context of a good match to a Kozak consensus start site. Searches of the nucleic acid databases with the coding region revealed two mouse EST clones with highly similar sequences (mEST995 and mEST896). These two sequences were identical to each other except for differences at their very 3′ ends, which probably arise by alternative splicing events. The sequence of mEST995 was used to design PCR primers, and the full-length sequence of the rat cDNA was amplified and cloned. The C-terminal portion of ZAP was cloned from a Rat2 cell cDNA library by PCR using sense primer CZAP-SP (5′GAGCTCTCTGGGCTTAACC-3′) and antisense primer CZAP-AP (5′ ATATAGGCGGCCGCCCTCTGGAC CTCTTCTCTTC-3′). The sense primer lies upstream from an internal NheI site in NZAP; the antisense primer introduces a NotI site (bolded) immediately downstream from the coding sequence to facilitate its cloning into the myc-tagged expression vector. PCR was conducted with Expand High Fidelity PCR kit (Roche) under the following conditions: 10 cycles of 94° C. for 15 seconds, 50° C. for 30 seconds, 72° C. for 120 seconds each cycle, followed by 20 cycles of 94° C. for 15 seconds, 55° C. for 30 seconds and 72° C. for 120+5 seconds each cycle. The PCR product was digested with NheI and NotI and then cloned into pcDNA4/TO2-NZAP-myc. The sequence of the complete cDNA contained 789 codons (sequence deposited in GenBank, accession # pending); the initial cDNA corresponded perfectly to the aminoterminal one-third of the sequence. The predicted amino acid sequences of the rat protein and of the similar mouse proteins contained a cluster of four unusual CCCH-type zinc fingers, previously found in only a few RNA-binding proteins (FIG. 3B). The gene was dubbed rZAP, for rat Zinc-finger Antiviral Protein, and the initial antiviral N-terminal fusion construct was named NZAP-zeo.

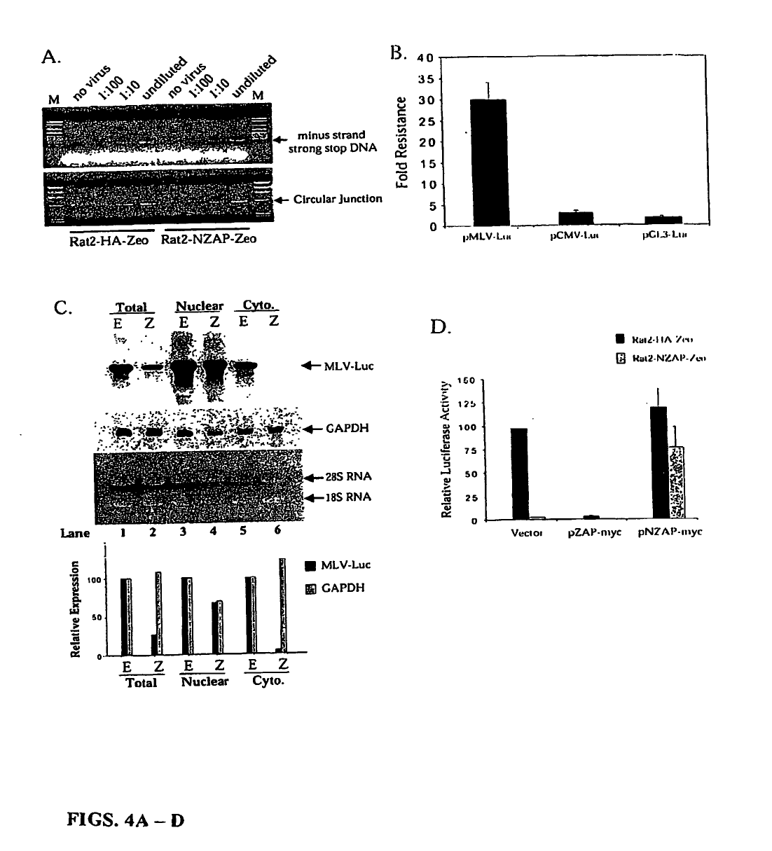

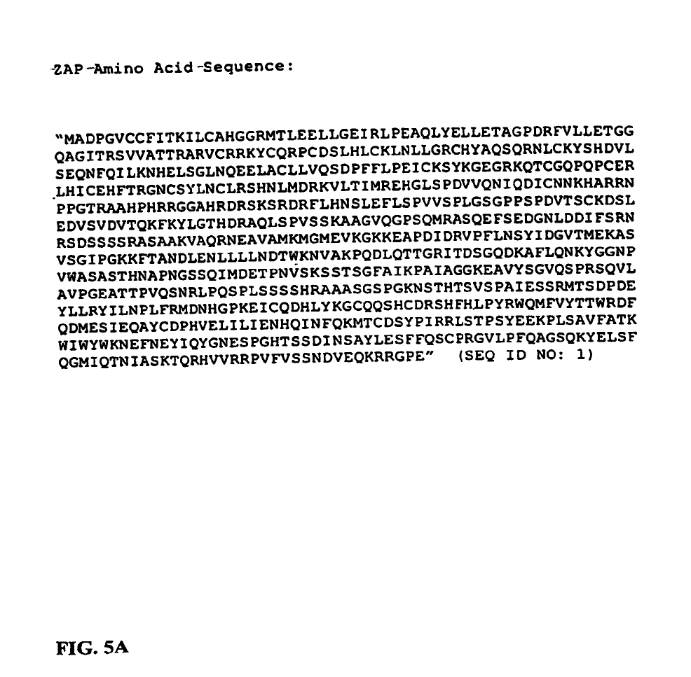

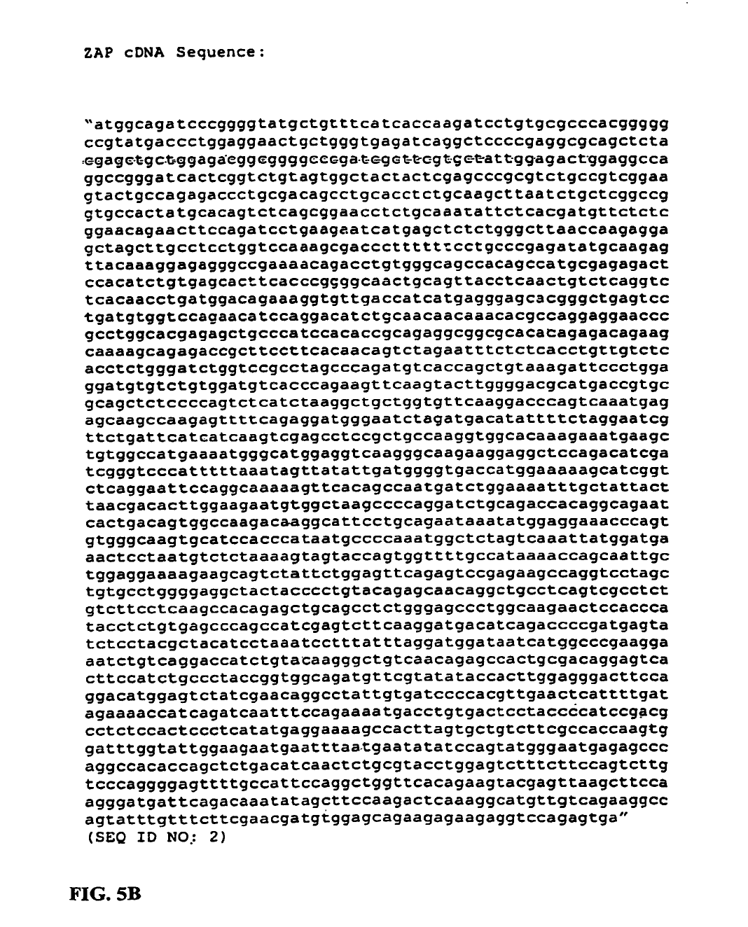

The resistance to virus transduction could be effected at many stages of infection. To determine the position of the block in the retrovirus life cycle, Rat2 cells expressing NZAP-zeo or the empty vector as control were acutely infected with Eco-Luc virus and the synthesis of viral DNA was examined by PCR. Comparable levels of minus strand strong stop DNA were synthesized in both lines, suggesting no block to entry or initiation of reverse transcription (FIG. 4A). Similar experiments with appropriate primers showed no defects in plus strand DNA synthesis. To test for nuclear entry of the viral DNA, the cells were infected with a similar vector, Eco-GFP, but with a higher titer, and circular viral DNAs were analyzed by PCR amplification of the LTR-LTR junction. Comparable levels of the circular forms were detected (FIG. 4A), suggesting no block to nuclear entry. To test for defects in viral gene expression, the retroviral Luc reporter DNA was directly introduced into the cells by lipid-mediated transformation, bypassing all the early stages of infection. The cells expressing NZAP-zeo expressed 30-fold lower luceriferase than the controls, the same decrease seen after retroviral infection (FIG. 4B). These results suggest that NZAP-zeo does not affect early events but rather acts to inhibit retroviral gene expression from the viral DNA. The effect was specific for retroviral expression, since expression of a control reporter construct via mRNAs lacking viral sequences was only reduced three fold in the NZAP-zeo line (FIG. 4B).

To determine the mechanism of the resistance to virus gene expression, viral mRNA levels in cells expressing NZAP-zeo or the empty vector were measured. After infection with Eco-Luc virus, total cellular RNAs were prepared, and in addition, cells were fractionated and nuclear and cytoplasmic RNAs were isolated. Cells of each line were infected with freshly collected Eco-Luc virus for at least five hours. Two days after infection the cells were trypsinized and collected for RNA preparation. Approximately 20% of the cells were used to extract total RNA with RNA extraction kit (Qiagen). The rest of the cells were used to extract cytoplasmic RNA with the the RNA extraction kit (Qiagen) following manufacturer's instructions. The nuclear pellet from cytoplasmic RNA extraction was washed twice with the lysis buffer and the nuclear RNA was then extracted as for total RNA. The RNAs were separated by gel electrophoresis, blotted, and probed with 32P-labelled luciferase sequences (FIG. 4C). The whole-cell preparations showed a modest reduction in level of the viral RNA in the NZAP-zeo cells as compared to the control cells. The fractionated RNAs, however, showed a dramatic result. The levels of the viral RNA in the nuclear fraction were virtually identical in the NZAP-zeo and control lines, suggesting that there was no significant effect on transcription initiation or elongation. But the cytoplasmic viral RNA was almost completely abolished in the NZAP-zeo cells, while high levels were found in the controls. Quantitation of longer exposures indicated a 30-fold reduction in the levels of the cytoplasmic RNA relative to the controls (FIG. 4C). The levels of rRNA and of a housekeeping mRNA, GAPDH, were unaffected. These results suggest that the NZAP-zeo protein specifically eliminated the cytoplasmic fraction of the viral RNA.

The antiviral construct as originally isolated contained only the 5′ one third of the complete rZAP coding region, fused to the zeocin resistance gene. The complete rZAP protein could have similar activity as the fusion protein; alternatively, the full protein could normally have a positive activity that is antagonized by a dominant negative activity of the fragment. To test the function of the full-length protein, the complete ZAP ORF was cloned into an expression plasmid under the control of the CMV promoter, tagged with a myc epitope at the carboxyterminus, forming pZAP-myc. The pZAP-myc was used to transform Rat2 cells expressing the empty vector control, and also those expressing NZAP-zeo, and the effects on Eco-Luc transduction were measured as before. The full-length protein induced a dramatic inhibition of viral vector expression on its own (FIG. 4D). The inhibition was even greater when rZAP and NZAP-zeo were expressed together. These results suggest that the full-length rZAP also acts to negatively regulate viral transcripts.

Other constructs were tested to look for forms of ZAP that would interfere with the antiviral activity of the wild-type protein. The 5′ portion of the gene present in NZAP-zeo was excised from the pBabe-HAZ vector and expressed without the zeo fusion partner and with a myc epitope tag. The NZAP fragment was excised from pBabe-NZAP-Zeo with EcoRI-NotI and cloned into pcDNA4/TO2-myc-HisB (Invitrogen) to generate a myc-tagged NZAP. The fragment reproducibly caused a small increase in the level of luciferase detected after infection by Eco-Luc virus (FIG. 4D). Moreover, this fragment almost completely suppressed the inhibition in cells containing NZAP-zeo or rZAP, restoring normal luciterase expression. Thus, this fragment antagonized the normal ZAP activity.

The results above show that direct selections for virus-resistant cells (14) can be used to identify new genes with potent antiviral activity. Key aspects to the selection were the use of highly infectible parental cells; transfer of a large cDNA library to the cells; repeated and saturating infection with a counterselectable virus; and the efficient killing of virus-sensitive cells. The gene recovered here, rZAP, is sufficient on its own to induce an antiviral state with no apparent affect on cell viability or physiology. The wild-type rZAP, as well as the truncated NZAP-zeo fusion protein, causes a profound inhibition of expression of reporter genes carried by retroviral vectors, acting at the level of the cytoplasmic viral RNA.

ZAP prevents the accumulation of cytoplasmic viral RNA because of the presence of the cluster of four unusual CCCH-type zinc fingers suggests that ZAP may interact directly with the viral RNA. These fingers are found in a small family of RNA binding proteins; the best-known member of the family is tristetraprolin (TTP), a protein which negatively regulates the levels of TNF-α (24) and GM-CSF mRNAs (25). TTP binds AU-rich sequences in the 3′ UTR of the TNF-a mRNA (24,26) and recruits the exosome to degrade the mRNA (27); it acts in opposition to the binding of HuR, another RNA-binding protein which stabilizes its target. rZAP may act in a similar way at sequences found in viral RNAs, and perhaps also in specific cellular mRNAs. Consistent with this notion, preliminary tests of ZAP mutants suggest that all of the finger motifs are crucial for its activity (data not shown). However, there is little sequence similarity to TTP outside the fingers, and the distinctive parts of the molecule may carry out other functions than the induction of RNA degradation.

The normal function of rZAP may be to regulate one or more specific cellular mRNAs. However, like PKR, RNase L, and the MX proteins, rZAP may be an example of a gene whose primary function will prove to be inhibiting viral gene expression and inducing an innate immunity to viral infection. The full range of viruses restricted by rZAP is not yet known. Preliminary results, however, suggest that the gene may have an extended antiviral activity; for example, rZAP can potently block replication of Sindbis virus in Rat2 cells (M. MacDonald, personal communication). Activation of expression of the endogenous gene could ultimately help induce immunity and protect individuals from disease caused by viral infections.

### REFERENCES

1. An, D. S. et al., J. Virol, Vol. 71, pgs. 1397-1404 (1997).
2. Bothe, G. W., Haspel, J. A., Smith, C. L., Wiener, H. H., Burden, S. J., Genesis, Vol. 26, pgs. 165-166 (2000).
3. Carballo, E., Lai, W. S., Blackshear, J. P., Blood, Vol. 95, pgs. 1891-1899 (2000).
4. Chen, C. Y. et al., Cell, Vol. 107, pgs. 451-464 (2001).
5. Dean, J. L., et al., Molecular & Cellular Biology, Vol. 21, pgs. 721-730 (2001).
6. Der, S. D., Yang, Y. L., Weissmann, Williams, B. R., Proceedings of the National Academy of Sciences of the United States of America, Vol. 94, pgs. 3279-3283 (1997).
7. Gao, G., Goff, S. P., Molecular Biology of the Cell, Vol. 10, pgs 1705-1717 (1999).
8. Haller, O., Frese, M., Kochs, G., Revue Scientifique et Technique, Vol. 17, pgs 220-230 (1998).
9. Kerr, I. M., Brown, R. E., Proceedings of the National Academy of Sciences of the United States of America, Vol. 75, pgs 256-260 (1978).
10. Lai, W. S. et al., Molecular & Cellular Biology, Vol. 19, pgs. 4311-4323 (1999).
11. Lai, W. S., Blackshear, P. J., Journal of Biological Chemistry, Vol. 276, pgs 2344-2354 (2001).
12. Landolofo, S., Gribaudo, G., Angeretti, A., Gariglio, M., Pharmacology & Therapeutics, Vol. 65, pgs 415-442 (1995).
13. Levy, D. E., Garcia-Sastre, A., Cytokine & Growth Factor Reviews, Vol. 12, pgs 143-156 (2001).
14. Markowitz, D., Goff, S. P., Bank, A., J. Virology, Vol. 62, pgs 1120-1124 (1988).
15. Morganstern, J. P., Land, H., Nucleic Acids Research, Vol. 18, pgs 3587-3596 (1990).
16. Naldini, L. et al., Science, Vol. 272, pgs 263-267 (1996).
17. Player, M. R., Torrence, P. F., Pharmacology & Therapeutics, Vol. 78, pgs 55-113 (1988).
18. Southern, P. J., Berg, P., J Mol Appl Genetics, Vol. 1, pgs 327-341 (1982).
19. Zhou, A., Paranjape, J. M., Der, S. D., Williams, B. R., Silverman, R. H., Virology, Vol. 258, pgs 435-440 (1999).

1. An isolated ZAP protein.

2. The protein of claim 1, wherein the protein is a human ZAP protein.

3. The protein of claim 1, wherein the protein is a rat ZAP protein.

4. The protein of claim 1, wherein the protein is a mouse ZAP protein.

5. The protein of claim 1, wherein the protein comprises the amino sequence set forth in SEQ ID NO: 1.

6. The protein of claim 1, wherein the protein has deleted from it a region which causes protein instability.

7. The protein of claim 6, wherein the region is the WWE region.

8. An isolated nucleic acid which encodes a ZAP protein.

9. The nucleic acid of claim 8, wherein the nucleic acid is DNA.

10. The DNA of claim 8, wherein the DNA is cDNA.

11. The cDNA of claim 10, wherein the cDNA comprises the nucleic acid sequence set forth in SEQ ID NO: 2.

12. The DNA of claim 9, wherein the DNA is a genomic DNA.

13. The nucleic acid of claim 8, wherein the nucleic acid is RNA.

14. The nucleic acid of claim 8, wherein the ZAP protein is a human ZAP protein.

15. The nucleic acid of claim 8, wherein the ZAP protein is a rat ZAP protein.

16. The nucleic acid of claim 8, wherein the ZAP protein is a mouse ZAP protein.

17. The nucleic acid of claim 8, wherein the protein has deleted from it a region which causes protein instability.

18. The nucleic acid of claim 17, wherein the region is the WWE region.

19. The nucleic acid of claim 8, wherein the nucleic acid is labeled with a detectable marker.

20-36. (canceled)

37. A method for increasing the amount of ZAP protein in a subject's cells which comprises administering to the subject an amount of ZAP protein effective to increase the amount of ZAP protein in the subject's cells.

38-56. (canceled)


SEQUENCE LISTING (10 sequences)

SEQ ID NO: 1 - protein, 776 aa, mammalian
MADPGVCCFITKILCAHGGRMTLEELLGEIRLPEAQLYELLETAGPDRFVLLETGGQAGITRSVVATTRARVCRRKYCQRPCDSLHLCKLNLLGRCHYAQSQRNLCKYSHDVLSEQNFQILKNHELSGLNQEELACLLVQSDPFFLPEICKSYKGEGRKQTCGQPQPCERLHICEHFTRGNCSYLNCLRSHNLMDRKVLTIMREHGLSPDVVQNIQDICNNKHARRNPPGTRAAHPHRRGGAHRDRSKSRDRFLHNSLEFLSPVVSPLGSGPPSPDVTSCKDSLEDVSVDVTQKFKYLGTHDRAQLSPVSSKAAGVQGPSQMRASQEFSEDGNLDDIFSRNRSDSSSSRASAAKVAQRNEAVAMKMGMEVKGKKEAPDIDRVPFLNSYIDGVTMEKASVSGIPGKKFTANDLENLLLLNDTWKNVAKPQDLQTTGRITDSGQDKAFLQNKYGGNPVWASASTHNAPNGSSQIMDETPNVSKSSTSGFAIKPAIAGGKEAVYSGVQSPRSQVLAVPGEATTPVQSNRLPQSPLSSSSHRAAASGSPGKNSTHTSVSPAIESSRMTSDPDEYLLRYILNPLFRMDNHGPKEICQDHLYKGCQQSHCDRSHFHLPYRWQMFVYTTWRDFQDMESIEQAYCDPHVELILIENHQINFQKMTCDSYPIRRLSTPSYEEKPLSAVFATKWIWYWKNEFNEYIQYGNESPGHTSSDINSAYLESFFQSCPRGVLPFQAGSQKYELSFQGMIQTNIASKTQRHVVRRPVFVSSNDVEQKRRGPE

SEQ ID NO: 2 - DNA, 2331 bp, mammalian
atggcagatcccggggtatgctgtttcatcaccaagatcctgtgcgcccacgggggccgtatgaccctggaggaactgctgggtgagatcaggctccccgaggcgcagctctacgagctgctggagacggcggggcccgatcgcttcgtgctattggagactggaggccaggccgggatcactcggtctgtagtggctactactcgagcccgcgtctgccgtcggaagtactgccagagaccctgcgacagcctgcacctctgcaagcttaatctgctcggccggtgccactatgcacagtctcagcggaacctctgcaaatattctcacgatgttctctcggaacagaacttccagatcctgaagaatcatgagctctctgggcttaaccaagaggagctagcttgcctcctggtccaaagcgacccttttttcctgcccgagatatgcaagagttacaaaggagagggccgaaaacagacctgtgggcagccacagccatgcgagagactccacatctgtgagcacttcacccggggcaactgcagttacctcaactgtctcaggtctcacaacctgatggacagaaaggtgttgaccatcatgagggagcacgggctgagtcctgatgtggtccagaacatccaggacatctgcaacaacaaacacgccaggaggaacccgcctggcacgagagctgcccatccacaccgcagaggcggcgcacacagagacagaagcaaaagcagagaccgcttccttcacaacagtctagaatttctctcacctgttgtctcacctctgggatctggtccgcctagcccagatgtcaccagctgtaaagattccctggaggatgtgtctgtggatgtcacccagaagttcaagtacttggggacgcatgaccgtgcgcagctctccccagtctcatctaaggctgctggtgttcaaggacccagtcaaatgagagcaagccaagagttttcagaggatgggaatctagatgacatattttctaggaatcgttctgattcatcatcaagtcgagcctccgctgccaaggtggcacaaagaaatgaagctgtggccatgaaaatgggcatggaggtcaagggcaagaaggaggctccagacatcgatcgggtcccatttttaaatagttatattgatggggtgaccatggaaaaagcatcggtctcaggaattccaggcaaaaagttcacagccaatgatctggaaaatttgctattacttaacgacacttggaagaatgtggctaagccccaggatctgcagaccacaggcagaatcactgacagtggccaagacaaggcattcctgcagaataaatatggaggaaacccagtgtgggcaagtgcatccacccataatgccccaaatggctctagtcaaattatggatgaaactcctaatgtctctaaaagtagtaccagtggttttgccataaaaccagcaattgctggaggaaaagaagcagtctattctggagttcagagtccgagaagccaggtcctagctgtgcctggggaggctactacccctgtacagagcaacaggctgcctcagtcgcctctgtcttcctcaagccacagagctgcagcctctgggagccctggcaagaactccacccatacctctgtgagcccagccatcgagtcttcaaggatgacatcagaccccgatgagtatctcctacgctacatcctaaatcctttatttaggatggataatcatggcccgaaggaaatctgtcaggaccatctgtacaagggctgtcaacagagccactgcgacaggagtcacttccatctgccctaccggtggcagatgttcgtatataccacttggagggacttccaggacatggagtctatcgaacaggcctattgtgatccccacgttgaactcattttgatagaaaaccatcagatcaatttccagaaaatgacctgtgactcctaccccatccgacgcctctccactccctcatatgaggaaaagccacttagtgctgtcttcgccaccaagtggatttggtattggaagaatgaatttaatgaatatatccagtatgggaatgagagcccaggccacaccagctctgacatcaactctgcgtacctggagtctttcttccagtcttgtcccaggggagttttgccattccaggctggttcacagaagtacgagttaagcttccaagggatgattcagacaaatatagcttccaagactcaaaggcatgttgtcagaaggccagtatttgtttcttcgaacgatgtggagcagaagagaagaggtccagagtga

SEQ ID NO: 3 - DNA, 78 bp, Artificial Sequence
  PCR Primer
ataagcttgccaccatggcttstccststgstgttccagatatgctgaattcggcggccgcgccaagttgaccagtgc

SEQ ID NO: 4 - DNA, 26 bp, Artificial Sequence
  PCR Primer
atatcgattcagtcctgctcctcggc

SEQ ID NO: 5 - DNA, 38 bp, Artificial Sequence
  Oligonucleotide
ctagataacttcgtataatgtatgctatacgaagttat

SEQ ID NO: 6 - DNA, 38 bp, Artificial Sequence
  Oligonucleotide
ctagataacttcgtatagcatacattatacgaagttat

SEQ ID NO: 7 - DNA, 25 bp, Artificial Sequence
  PCR Primer
gcttatccatatgatgttccagatt

SEQ ID NO: 8 - DNA, 34 bp, Artificial Sequence
  PCR Primer
atataggcggccgccctctggacctcttctcttc

SEQ ID NO: 9 - DNA, 19 bp, Artificial Sequence
  PCR Primer
gagctctctgggcttaacc

SEQ ID NO: 10 - DNA, 34 bp, Artificial Sequence; identical to SEQ ID NO: 8
  PCR Primer